(19) United States
(12) Patent Application Publication
(10) Pub. No.: US 2026/0276630 A1
(43) Pub. Date: Sep. 17, 2026

(54) SURFACE ACOUSTIC WAVE FACILITATED FLUIDIC ASSAY SYSTEMS AND METHODS

(71) Applicant: The Regents of the University of California, Oakland, CA (US)

(72) Inventors: James R. Friend, San Diego, CA (US); Lei Zhang, San Diego, CA (US); Shuai Zhang, La Jolla, CA (US); Cecile Floer, La Jolla, CA (US)

(21) Appl. No.: 19/080,325

(22) Filed: Mar. 14, 2025

(51) Int. Cl. G01N 33/53; G01N 29/14

(52) U.S. Cl. CPC G01N 33/5306; G01N 29/14; G01N 33/5304; G01N 2291/0255

(57) ABSTRACT

Surface acoustic wave facilitated fluidic assay systems and methods are disclosed. An example system includes a piezoelectric substrate; a transducer assembly including a plurality of transducers that are deposited on the piezoelectric substrate, the transducer assembly configured to apply alternating electric fields to the piezoelectric substrate to induce surface acoustic waves; and a well plate configured to be positioned to receive the surface acoustic waves from the piezoelectric substrate, the well plate including a plurality of wells configured to contain fluid samples, in which the plurality of transducers are arranged to align with the plurality of wells to allow the surface acoustic waves to propagate into the fluid samples in the wells, thereby facilitating performance of an assay protocol performed in the fluid samples.

### STATEMENT REGARDING FEDERALLY SPONSORED RESEARCH OR DEVELOPMENT

This invention was made with government support under NS115591 awarded by the National Institutes of Health. The government has certain rights in the invention.

### TECHNICAL FIELD

The present disclosure relates to fluidic assays, and more particularly to a surface acoustic wave (SAW) facilitated fluidic assay systems and methods.

### BACKGROUND

Enzyme-linked immunosorbent assays (ELISAs) are widely used analytical techniques in biology and clinical diagnostics. These assays rely on antigen-antibody interactions to detect and quantify specific molecules in complex biological samples. ELISAs have applications across various fields including medical research, disease diagnosis, environmental monitoring, and pharmaceutical development. The basic ELISA protocol involves several steps such as coating a surface with capture antibodies, blocking non-specific binding sites, adding samples containing target molecules, and detecting bound molecules using enzyme-linked detection antibodies.

### SUMMARY

An aspect of the present document relates to a surface acoustic wave (SAW) enhanced enzyme-linked immunosorbent assay (ELISA) system. In some embodiments, the system includes a piezoelectric substrate; a transducer assembly including a plurality of transducers that are deposited on the piezoelectric substrate, the transducer assembly configured to apply alternating electric fields to the piezoelectric substrate to induce surface acoustic waves; and a well plate configured to be positioned to receive the surface acoustic waves from the piezoelectric substrate, the well plate including a plurality of wells configured to contain fluid samples relating to an ELISA protocol, in which the plurality of transducers are arranged to align with the plurality of wells to allow the surface acoustic waves to propagate into the fluid samples in the wells, thereby facilitating performance of the ELISA protocol.

Another aspect of the present document relates to a method for facilitating a fluid assay. In some embodiments, the method includes providing a transducer assembly that comprises at least one transducer deposited on a piezoelectric substrate; positioning a well plate containing at least one well with a fluid sample on the piezoelectric substrate; applying an alternating electric field to the transducer assembly to induce surface acoustic waves in the piezoelectric substrate; and allowing the surface acoustic waves to propagate into the fluid sample to facilitate an assay protocol performed in the fluid sample. In some embodiments, the fluidic assay is an ELISA assay, or another fluidic assay as disclosed herein.

A further aspect of the present document relates to an enzyme-linked immunosorbent assay (ELISA) system. In some embodiments, the system includes: a microplate reader; and a surface acoustic wave (SAW) module configured to be positioned beneath a standard multi-well plate. The SAW module includes: a piezoelectric substrate; and a transducer assembly comprising at least one transducer deposited on the piezoelectric substrate and configured to apply alternating electric fields to induce surface acoustic waves. The system is configured to propagate the surface acoustic waves into fluid samples in a standard multi-well plate when the standard multi-well plate is positioned on the SAW module during operation to facilitate or enhance reaction kinetics, including binding kinetics, of an ELISA protocol.

A still further aspect of the present document relates to an adapter for facilitating a fluidic assay. In some embodiments, the adapter includes a piezoelectric substrate configured to be acoustically coupled to a reaction vessel; a transducer assembly comprising at least one transducer that is deposited on the piezoelectric substrate; and a signal interface configured to connect to a power source. The adapter is configured to generate surface acoustic waves that propagate into a fluid sample in the reaction vessel to enhance reaction kinetics of the fluidic assay. In some embodiments, the adapter is configured to be incorporated into an existing assay equipment without or with minimal modification of the existing assay equipment.

### BRIEF DESCRIPTION OF FIGURES

Non-limiting and non-exhaustive examples are described with reference to the following figures.

FIG. 1 illustrates a block diagram of a surface acoustic wave (SAW) facilitated enzyme-linked immunosorbent assay (ELISA) system, according to some embodiments of the present disclosure.

FIG. 2 illustrates a process of using surface acoustic waves (SAW) to facilitate a fluidic assay, according to some embodiments of the present disclosure.

FIG. 3A illustrates an overview of the experimental setup, according to some embodiments of the present disclosure.

FIG. 3B illustrates an ELISA protocol sequence, according to some embodiments of the present disclosure.

FIGS. 3C and 3D are a top-down view and an expanded view of a transducer assembly, according to some embodiments of the present disclosure.

FIG. 4 illustrates a bar graph comparing binding efficiency for different SAW frequencies, according to some embodiments of the present disclosure.

FIG. 5A illustrates absorbance curves after 1 minute of incubation with and without SAW treatment, according to some embodiments of the present disclosure.

FIG. 5B illustrates absorbance values over time for SAW-treated and control samples, according to some embodiments of the present disclosure.

FIG. 6A illustrates absorbance measurements over time comparing SAW and non-SAW conditions, according to some embodiments of the present disclosure.

FIG. 6B illustrates absorbance measurements under different experimental conditions, according to some embodiments of the present disclosure.

FIG. 7 illustrates a graph comparing absorbance measurements for SAW and non-SAW conditions at different time points, according to some embodiments of the present disclosure.

FIG. 8 illustrates absorbance indicated binding versus target antigen concentration with and without SAW, according to some embodiments of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to fluidic assays, such as enzyme-linked immunosorbent assay (ELISAs), and more particularly to a surface acoustic wave (SAW) facilitated fluidic assays for improving assay speed and sensitivity.

Enzyme-linked immunosorbent assays (ELISA) are widely used in biology and clinical diagnosis. Relying on antigen-antibody interaction through diffusion, the standard ELISA protocol can be time-consuming, preventing its use in rapid diagnostics. Surface acoustic waves (SAW) may be used to enhance performance by achieving a time-saving and more sensitive ELISA without changing the standard set-up and protocol. Each step of at least a portion of the assay, from the initial antibody binding onto the walls of the well plate to the target analyte molecules' binding for detection-except, notably, for the blocking step—is improved principally via acoustic streaming-driven advection. Using SAW, the time needed for one step of an example ELISA may be reduced from 60 min to 15 min to achieve the same binding amount. By extending the duration of SAW exposure to 20 min, the sensitivity can be significantly improved over the 60-min, 35 deg. C. ELISA without SAW. SAW may confer beneficial improvements to bead-based ELISA by combining it with SAW to further reduce the time needed for binding to 2 min. By significantly increasing the speed of ELISA, its utility may be improved for a wide range of point-of-care diagnostics applications.

In some embodiments, assays are common across many disciplines, including biology, chemistry, clinical diagnostics, environmental monitoring, and pharmaceutical research. Most are designed to detect and quantify proteins, nucleic acids, hormones, enzymes, and similar biomolecules in addition to non-biological entities like toxins and environmental contaminants. The detection principles vary according to the target. Polymerase chain reaction (PCR) assays are the best known among them. Though very popular, PCR assays solely serve to detect deoxyribonucleic acid (DNA). Surface plasmon resonance (SPR) and isothermal titration calorimetry (ITC) assays identify the interaction between two or more molecules, such as receptor-ligand binding or protein-protein binding. There are also assays that involve the separation of analytes based on size, charge, or other properties, such as the western blot (WB). Among all these assays, the immunoassay stands out as one of the most frequently used methods because of its versatility, sensitivity, scalability, cost efficiency, and ease of use. Immunoassays take advantage of the specific interaction between biological reagents, typically antigens and antibodies, to selectively detect the presence and concentration of target analytes in complex biological mixtures.

The detection and quantification of target molecules at low concentrations have historically been a significant challenge in immunoassay development. Indeed, others show from calculations that without directed transport of biomolecules, individual nanoscale sensors may be limited to picomolar-order sensitivity for practical time scales. Sensing low concentrations is desirable, as many biological and clinical applications need the measurement of analytes present at trace levels, ideally in a simple and fast protocol. Building upon competitive binding principles from earlier studies, others developed the radioimmunoassay (RIA) by introducing radioactively-labeled insulin into a plasma sample, enabling indirect determination of the unlabeled insulin concentration through competition for antibody binding and subsequent radioactivity measurement. The development of RIA revolutionized clinical diagnostics and research by providing a highly sensitive, specific, and reproducible method for measuring low concentrations of analytes in complex biological samples. However, the use of radioactive isotopes raised concerns about safety, prompting a search for alternative labeling methods and detection techniques. The enzyme-based immunoassay (ELISA) was developed in response; besides offering a non-ionizing radiation-based assay, it also provided a longer shelf life than RIA. The ELISA employs enzyme-linked reactions to amplify the detection signal, making it both highly sensitive and specific. Fluorescent immunoassays (FIA) and chemiluminescent immunoassays (CLIA) are also popular assays today, detecting analytes through fluorescent labeling and light emission from the chemical reactions, respectively. However, ELISA offers a simple and easy-to-read colorimetric output for quantified detection of the target analyte.

Enzyme-based immunoassays can be used to detect a wide range of analytes, including proteins, antibodies, and hormones. Compared to lateral flow immunoassays (LFIA) that have been commonplace since the 1970s in their utilization for pregnancy tests and most recently the near-ubiquitous use in home diagnosis of COVID, ELISA offers far greater sensitivity and reproducibility with the same general assay conditions and reagents. In studies by others, the sensitivity of LFIA has been improved to nearly compete with ELISA-under certain conditions and for specific assay targets. CRISPR-based (CAS-12/CAS-13a) assays have become popular, though there remain questions as to their actual sensitivity. The wide range of concentration that may be repeatably quantified using ELISA is another reason it is a popular assay, as is the fact it can be performed using parallel, high throughput methods using microplate readers and automated processing equipment. While PCR is another popular method for DNA detection that is likewise automated in many applications, the cost of ELISA is significantly lower: PCR analysis needs specialized reagents, such as Taq polymerase and dNTPs, and specialized equipment, such as thermal cyclers. Though researchers have made progress in lowering the cost and effort of PCR through microfluidics, ELISA remains less expensive.

More generally, microfluidics has had a beneficial impact in assay quality and cost reduction, reducing sample sizes and reagent consumption, and reducing the time and effort needed in completing assays. The adoption of soft, easily sealed, and rapidly reproducible materials (polydimethylsiloxane, PDMS) and paper-based methods have continued this trend toward better assays for lower costs. For the same reasons, miniaturization techniques have also been improved, thus allowing the integration of micropumps and micromixers into devices. More recently, acoustofluidics has helped to overcome one of the key remaining problems in microfluidics used for lab-on-a-chip applications: the propulsion and manipulation of fluids and suspended objects within. There have been demonstrations of its use with PDMS and paper, and a number of groups have spent the past decade expanding their understanding and creative use of the phenomena.

However, one problem that microfluidics and acoustofluidics have not helped with—at least in ELISA—is its slowness. It takes from 3 h to 12 h to complete an ELISA: every binding step from the initial antibody binding to the target analyte binding is time consuming. For this reason, LFIA is also far more common: there are less sensitive versions of LFIA that only take 15 min. If ELISA was faster, it might provide a much better test for the detection of disease without the drawbacks of having to wait so long and reducing the user's worry about potential false positive or negative results.

One of the attractive aspects of acoustofluidics, for example, is its ability to significantly speed up phenomena that rely on diffusion. For example, the charging of batteries relies on the diffusion of lithium ions within the electrolyte to replace those ions deposited on the anode and lost from the electrolyte; diffusion is the rate-limiting phenomenon preventing faster charging. Acoustic streaming-driven convection was discovered to convect lithium ions and overcome diffusion limitations, thereby allowing complete and rapid recharging of batteries in only minutes. The analytes in ELISA likewise need to diffuse in order to produce the binding necessary for the assay to complete, again representing the rate-limiting phenomenon preventing improvements in the speed of the assay in every binding step. More generally, surface acoustic waves may be used for mixing purposes in biosensors, to speed up the process, among other things.

The present disclosure provides a SAW-enhanced ELISA, using acoustic streaming to drive advection of the binding and target analyte molecules in each step of the assay. The goal is to accelerate the protein binding to decrease the ELISA time. Additionally or alternatively, the SAW may increase the sensitivity of the reaction. There is research reporting the use of SAW for detection improvement, however, they are done with droplets or PDMS channels directly on the transducer which is inconvenient for application.

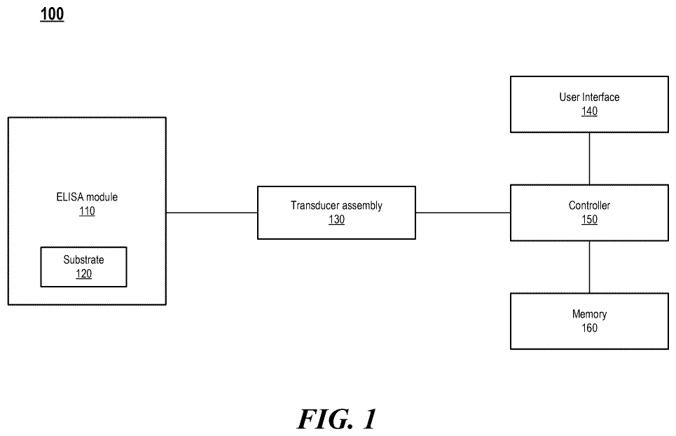

FIG. 1 shows a block diagram of a surface acoustic wave (SAW) facilitated enzyme-linked immunosorbent assay (ELISA) system 100, according to some embodiments of the present disclosure. The system 100 includes several interconnected components that work together to enhance ELISA protocols and other fluidic assays through the application of surface acoustic waves.

The ELISA module 110 is configured to perform ELISA assays. The ELISA module 110 is configured to hold a multi-well plate where fluid samples may be placed. The ELISA module 110 is acoustically coupled to a piezoelectric substrate 120. In some embodiments, the substrate 120 includes lithium niobate, e.g., a 127.86° Y-rotated cut. The substrate 120 has a thickness of, e.g., approximately 500 micrometers. In some embodiments, the substrate 120 is configured to generate Rayleigh surface acoustic waves.

The transducer assembly 130 is operably coupled to the substrate 120 and configured to induce the substrate 120 to produce surface acoustic waves, which in turn propagate to fluid samples placed in the multi-well plate in the ELISA module 110. The transducer assembly 130 may operate at frequencies between 5 MHz and 2 GHz. For example, for ELISA applications, the transducer assembly 130 operates at in the order of 10 MHz or 100 MHz, e.g., approximately 40 MHz, 60 MHz, 100 MHz, 120 MHz, 150 MHz. The transducer assembly 130 can operate in a pulsed mode with a variable duty cycle to control the ratio of heating to acoustic streaming in the fluid sample, e.g., using pulse width modulation with an adjustable duty cycle. The pulsed mode includes alternating between periods when the IDT is energized and periods when it is not energized. For example, the duty cycle is adjustable between 5% and 100%.

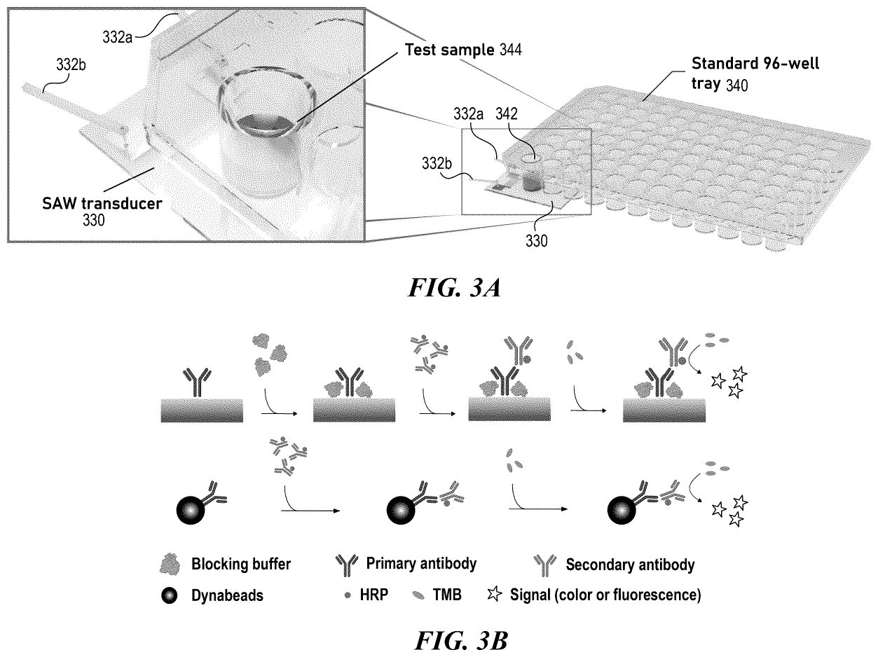

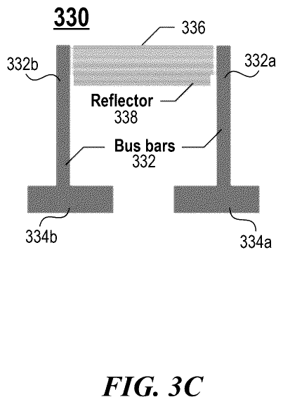

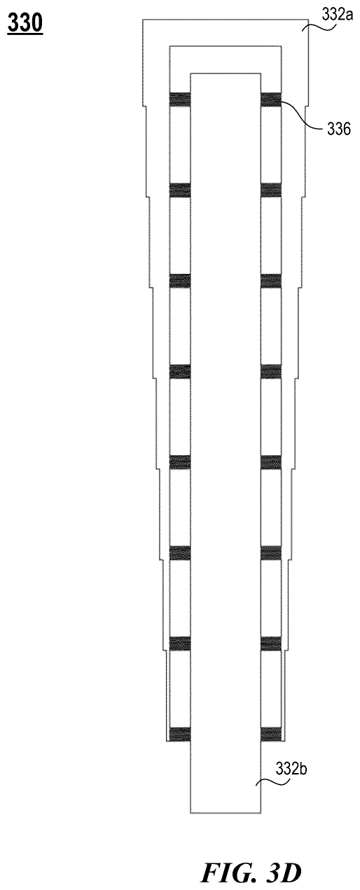

In some embodiments, the transducer assembly 130 includes at least one interdigital transducer (IDT) deposited on the piezoelectric substrate 120. The IDT is configured to apply alternating electric fields to induce surface acoustic waves. The IDT includes a plurality of electrode fingers arranged in an interdigitated pattern and includes a pair of bus bars with the electrode fingers extending between them. See, e.g., FIGS. 3B and 3D. The first bus bar is connected to a signal source while the second bus bar is connected to ground. The bus bars may be configured to maintain uniform electrical impedance across the plurality of electrode fingers. For example, one or both of the bus bars have varying cross-section areas along the length(s) of the bus bar(s), e.g., a stepped structure as illustrated in FIG. 3D, to maintain uniform electrical impedance across the plurality of electrode fingers.

In some embodiments, the IDT further includes at least one reflector positioned to direct the surface acoustic waves in a predetermined direction. A reflector may include one or more electrode fingers that are coupled to only one of the first bus bar or the second bus bar. Additional description of the IDT may be found elsewhere in the present document at, e.g., FIGS. 3B and 3D and the description thereof.

During operation, a well plate (see, e.g., FIG. 3A) is positioned within the ELISA module 110 to receive surface acoustic waves from the piezoelectric substrate 120. The well plate includes at least one well for containing a fluid sample. For example, the well plate is a standard 96-well plate with 96 wells arranged in a two-dimensional array (e.g., an 8 by 12 array). A plurality of transducers of the transducer assembly 130 are arranged to align with the plurality of wells such that one of the plurality of transducers (e.g., 336 in FIG. 3B or 3D) is positioned in operable proximity to one of the plurality of wells of the well plate. The operable proximity ensures that surface acoustic waves induced by a transducer substantially propagate into a corresponding well (e.g., a fluid sample in the corresponding well). In some embodiments, the transducer assembly 130 may be positioned underneath the well plate with individual transducers aligned beneath their corresponding wells. For example, each of the plurality of the transducers may be positioned at a particular distance from a corresponding well of the plurality of wells to allow the corresponding well to receive surface acoustic waves induced by the corresponding transducer. The piezoelectric substrate 120, together with an acoustic coupling medium in some embodiments, is interposed between the transducer assembly 130 and the bottom surface of the well plate to facilitate efficient acoustic energy transfer.

A transducer may include multiple finger pairs, e.g., 24 finger pairs, which are electrically connected to alternate bus bars. Additional description regarding a transducer may be found elsewhere in the present document. See, e.g., FIGS. 3B and 3D and the description thereof.

The well plate is positioned above the piezoelectric substrate and includes a bottom surface that receives the surface acoustic waves. The bottom surface may be positioned at a separation distance from the substrate that enables efficient transfer of acoustic energy into the fluid sample. An acoustic coupling medium, such as an acoustic gel or fluid, may be disposed between the piezoelectric substrate 120 and the well plate to facilitate transmission of surface acoustic waves while minimizing energy loss. The well plate is removeably positioned relative to the piezoelectric substrate 120 to allow for replacement.

The controller or controller circuit 150 (referred to as controller for brevity) is operably connected to the transducer assembly 130 and acts as a signal generator configured to provide electrical signals to the transducer assembly 130 (e.g., IDT). For a pulsed operation, the controller 150 can adjust the duty cycle between 5% and 100%. The controller 150 can synchronize operation of the components relating to the SAW function (collectively referred to as a SAW module, as described further below) with steps of the ELISA protocol and selectively activate the SAW module during at least some portions of the ELISA protocol, e.g., one or more of the binding steps but not during a blocking step. It can also adjust operating parameters based on the type of assay being performed.

Although the controller 150 is connected to the transducer assembly 130 in FIG. 1, this is for illustration purposes only and not intended to be limiting. The controller 150 may be operably coupled to the ELISA module 110 to control and/or monitor the operation therein. For example, the controller 150 may be part of a control system that includes a separate controller or control circuit designated to the ELISA module 110, and at least some of the controllers or control circuits in the control system may communicate to coordinate operations of the system 100.

The user interface 140 is operably connected to the controller 150 and provides an interface for users to interact with the system, adjust settings, and monitor progress. The memory 160 stores operational parameters, protocols, and data related to the various assays that can be performed. The user interface 140 may include a keyboard, a screen, a display device, a touch-screen, or the like, or a combination thereof.

The SAW module may include the substrate 120 and the transducer assembly 130. The SAW module is designed to be adaptable and can function in different configurations. For example, the SAW module is removable and can be inserted into an existing ELISA system without modification to the microplate reader. In some embodiment, the system includes a microplate reader and a SAW module configured to be positioned underneath (or otherwise operably coupled) to a standard multi-well plate. The system may include an acoustic coupling medium applicator and can be dimensioned to match the footprint of a standard multi-well plate. In some configurations, the SAW module is integrated into a tray or platform that replaces a standard tray of the microplate reader. The SAW module is configured to propagate the surface acoustic waves into fluid samples in the standard multi-well plate when the standard multi-well plate is positioned on the SAW module during operation to enhance binding kinetics of an ELISA protocol. The ELISA system includes a control system configured to synchronize operation of the SAW module with steps of the ELISA protocol, and selectively activate the SAW module during at least a portion of the ELISA protocol, e.g., one or more binding steps of the ELISA protocol.

Some embodiments of the present disclosure relate to an adapter for enhancing various fluidic assays beyond ELISA. The adapter includes the piezoelectric substrate with the IDT and a signal interface for connecting to a power source. It is configured to be acoustically coupled to various types of reaction vessels including multi-well plates, microfluidic chips, petri dishes, flasks, test tubes, or cuvettes. The adapter is configured to generate surface acoustic waves that propagate into a fluid sample in an acoustically coupled reaction vessel to enhance reaction kinetics. The adapter may include additional features such as a temperature sensor, alignment features, and a dispenser for automatically applying acoustic coupling medium. The adapter can enhance processes including ELISA, PCR, cell culture, crystallization, separation or purification, Clustered Regularly Interspaced Short Palindromic Repeats (CRISPR) and CRISPR-associated (CRISPR-Cas) based assays, aptamer-based assays and bead-based assays. For bead-based assays, the surface acoustic waves may prevent agglomeration of beads within the fluid sample.

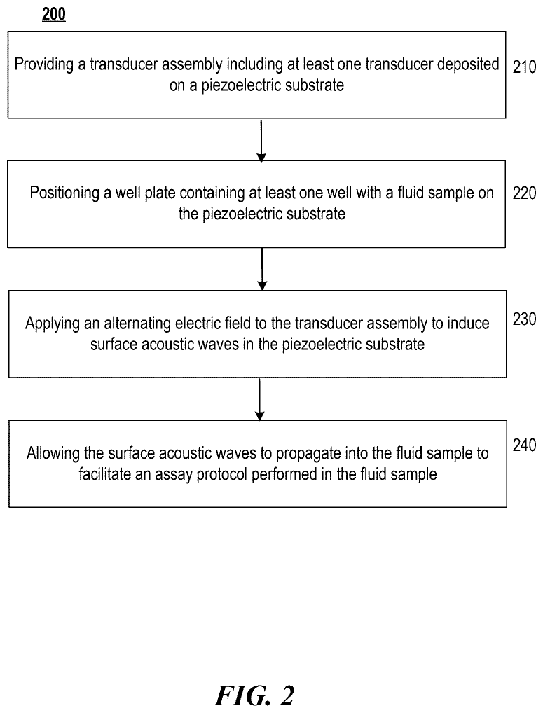

FIG. 2 illustrates a process of using surface acoustic waves (SAW) to facilitate a fluidic assay, according to some embodiments of the present disclosure.

At 210, the process 200 begins with providing a transducer assembly (e.g., 130). The transducer assembly includes at least one transducer deposited on a piezoelectric substrate (e.g., 120). The transducer may be an interdigital transducer (IDT) that is formed on the piezoelectric substrate through, e.g., standard fabrication techniques. In some embodiments, the piezoelectric substrate includes lithium niobate, e.g., a specially oriented cut that optimizes surface acoustic wave generation.

At 220, the process 200 proceeds to positioning a well plate containing at least one well with a fluid sample on the piezoelectric substrate. The fluid sample may relate to an ELISA protocol. An acoustic coupling medium, such as an acoustic gel or fluid, may be provided between the piezoelectric substrate and the well plate to facilitate efficient transmission of the acoustic waves from the substrate to the well plate. The well plate may be a standard multi-well plate format, such as a 96-well plate, that is commonly used in laboratory settings.

At 230, the process 200 includes applying an alternating electric field to the transducer assembly to induce surface acoustic waves in the piezoelectric substrate. The alternating electric field may operate the transducer assembly at a frequency between 5 MHz and 2 GHz, e.g., at approximately 60 MHz for ELISA enhancement. In some embodiments, the alternating electric field is applied in a pulsed mode with a variable duty cycle to control the ratio of heating to acoustic streaming in the fluid sample. The pulsed mode may employ pulse width modulation with an adjustable duty cycle, e.g., between 5% and 100%, depending on one or more factors including the desired balance between heating and acoustic effects. For example, the pulsed mode employs pulse width modulation with an adjustable duty cycle of 20%, 30%, 40%, 50%, 60%, 80%, etc. In some use cases, the applied electric field induces Rayleigh surface acoustic waves in the piezoelectric substrate. At least one reflector positioned adjacent to the IDT may be used to direct the surface acoustic waves in a predetermined direction.

At 240, the process 200 includes allowing the surface acoustic waves to propagate into the fluid sample to facilitate an assay protocol (e.g., the ELISA protocol) performed in the fluid sample. The propagation of surface acoustic waves generates both acoustic streaming and heating within the fluid sample, which significantly enhances the reaction kinetics. In some cases, this enhancement enables the ELISA protocol to be completed in less than 90 minutes, reducing the time by at least 50% compared to a standard ELISA protocol performed without surface acoustic waves.

In an exemplary implementation of the process, the ELISA protocol includes: binding a primary antibody to a surface of the well while applying the surface acoustic waves; adding a blocking buffer to the well; adding a sample containing target analyte to the well while applying the surface acoustic waves; adding a detection antibody to the well while applying the surface acoustic waves; and adding a substrate solution to the well to generate a detectable signal. The surface acoustic waves may be selectively applied to one or more of the steps. For example, the surface acoustic waves may be selectively applied during a primary antibody binding step, an analyte binding step, and a secondary antibody binding step. In some cases, surface acoustic waves are not applied during a blocking step. The process 200 may be applied to various types of ELISA protocols, including bead-based ELISA using functionalized microbeads or nanobeads. When nanobeads are used, the surface acoustic waves prevent agglomeration of the nanobeads, providing additional benefits to the assay performance.

The process 200 may further include measuring the detectable signal to determine a presence or concentration of the target analyte.

The following description includes example implementations in the context of ELISA assays for illustrative purposes only and is not intended to be limiting. The exemplary implementations disclosed herein use 96-well plates which are widely used in ELISA. The experimental set-up and protocol are provided in the following sections. Results achieved with the enhanced device are compared to those obtained with the standard ELISA process.

In exemplary implementations, double-side polished 127.68° Y-rotated cut lithium niobate (LiNbO₃) with a thickness of 500 μm was used as a substrate for the SAW device. A single port resonator with a wavelength of λ=100 μm, 65 μm, or 40 μm (i.e., f=40 MHz, 60 MHz, or 100 MHz, respectively) was fabricated upon it to generate Rayleigh SAW. Individual 400 nm-thick aluminum interdigital transducers (IDTs) were patterned with 24 finger pairs and 48 reflectors on one side using a standard lithography and lift-off process. The Rayleigh SAW was generated by applying a sinusoidal electric signal to the IDTs at the resonance frequency using a signal generator (WF1967 200 MHz single channel multifunction generator, NF Corporation, Yokohama, Japan) and an amplifier (403LA, Electronics & Innovation, Ltd., Rochester, NY, USA) at up to 0.8 W of power. The 96-well plate was laid on top of the substrate and the wave was transmitted to the liquid inside the well thanks to a 5 μL acoustic gel droplet placed between the substrate and the plate (see FIGS. 3A-3C).

FIG. 3A is an overview of the experimental setup according to some embodiments of the present disclosure. The test well 342 in a multi-well plate 340 is 6 mm in diameter containing 200 μL of reagent solution 344, filling the well to a 6 mm depth. FIG. 3B is the complete ELISA protocol used in this exemplary implementation; the SAW was used in the introduction of the primary antibody, secondary antibody, and analyte. The reason it was not used for the introduction of the blocking buffer is explained in the results.

FIG. 3C shows a top view of a transducer assembly 330, according to some embodiments of the present disclosure. The transducer assembly 330 includes a pair of bus bars 332, including a first bus bar 332a and a second bus bar 332b. These bus bars 332 serve as the electrical connection points for the assembly 330, where one bus bar (e.g., 332a) is connected to a signal source while the other bus bar (e.g., 332b) is connected to ground. The bus bars 332 are connected to pads 334a and 334b on one end, which provide larger surface areas for establishing electrical connections to external signal sources. The transducer assembly 330 may include a reflector 338 positioned to direct the acoustic waves in a predetermined direction. The reflector 338 includes electrically disconnected electrode fingers (all connected to one bus bar but not the other bus bar) that help guide the acoustic waves generated when the piezoelectric substrate is excited. Element 336 represents a transducer, which is positioned between the two bus bars and serves as the active element that applies alternating electric fields to the piezoelectric substrate to induce surface acoustic waves.

FIG. 3D provides an expanded view of the complete transducer assembly 330, according to some embodiments of the present disclosure. The bus bars 332a and 332b run along opposite sides of the transducer assembly 330. The transducer assembly 330 includes multiple transducers 336, which are arranged in a pattern between the bus bars 332. Each transducer 336 may include a set of interdigitated electrode fingers (e.g., 24 finger pairs) that apply alternating electric fields to specific regions of a piezoelectric substrate (e.g., 120). These transducers 336 are arranged to align with corresponding wells in a multi-well plate 340, such that the surface acoustic waves generated by each transducer 336 propagate primarily into its corresponding well. The spacing and arrangement of these transducers 336 determine the operating characteristics of the device, including its frequency response and wave propagation patterns.

The bottom surface of the well plate 340 may be positioned relative to the piezoelectric substrate at a separation distance that enables efficient transfer of acoustic energy into the fluid sample 344. The separation distance is occupied by an acoustic coupling medium. The acoustic coupling medium may be an acoustic gel or fluid.

Together, the components of the transducer assembly 330 work in concert to apply controlled alternating electric fields to the piezoelectric substrate, which then generates surface acoustic waves that propagate into the fluid samples in the wells of the plate 340 positioned above the assembly 330. This configuration allows for efficient transfer of acoustic energy into multiple fluid samples simultaneously, enhancing the ELISA or other fluidic assays performed in the wells.

To produce improved or superior mixing from the acoustic streaming, the acoustic wave in a fluid sample may be attenuated over a length scale comparable to the depth of the fluid sample in order to avoid reflection from the distal boundary, thus forming a traveling wave in support of bulk acoustic streaming from the source. A desirable frequency to use for the SAW may be determined by equating the attenuation length of the acoustic wave to the size of the fluid sample to be manipulated. Here, in some exemplary implementations, the reagent depth in each of the 96 wells of the tray was found to be 6 mm after introducing the fluid sample (explained in the following section). Using an expression for the frequency according to this condition,

$${f}_{β} = \sqrt{( {ρ c}_{saw}^{3} ) / [ 4 {π}^{3} ( μ + {μ}^{′} ) L ]} , \qquad ( 1 )$$

where ρ is the fluid density, c_{saw} is the speed of the surface acoustic wave in the lithium niobate substrate as 3900 m/s, L=6 mm is the length scale over which the acoustic wave needs to be attenuated, and μ and μ′ are the bulk and shear viscosities for the fluid, respectively. Using this equation, the approximate frequency was identified to be 100 MHz.

The simplistic equation (1) presumes there is no other media along the acoustic wave's propagation path. Here, there is also 1 mm of polystyrene plastic (or another material, e.g., glass) of the bottom of the well plate increasing the attenuation, thus likely reducing the suitable frequency from the estimate produced from equation (1). The length scale may be longer to compensate for the presence of the attenuating plastic. With this in mind, two additional frequencies, 40 and 60 MHz, were tested in addition to the 100 MHz predicted by the equation, and measured the fluid velocity in the well induced by SAW at the same amplitude in the substrate at each of these three frequencies. The particle velocity of the lithium niobate substrate was measured via scanning laser Doppler vibrometry (UHF-120, Polytec, Waldbrönn, Germany) and set to 96 mm/s for all three frequencies by adjusting the input signal amplitude in the test implementations.

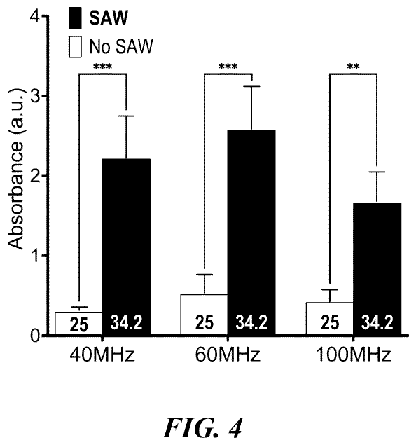

Then, 0.2 μm fluorescent microspheres (Fluoresbrite YG Carboxylate Microspheres 16592-1, Polysciences, Warrington, PA, USA) were used to track the flow while operating the SAW device at 96 mm/s at 40, 60, or 100 MHz using a high-speed camera (Fastcam Mini UX100, Photron, San Diego, CA, USA). These particles are sufficiently small to avoid direct acoustic forces upon them. According to the average fluid flow velocity given by PIVlab, the flow velocity in the well provided by the 40, 60, and 100 MHz devices were 0.009, 0.004, and 0.0002 m/s, respectively. This illustrates the effect of the attenuating plastic along the acoustic wave propagation path: 40 and 60 MHz acoustic waves produced streaming flows an order of magnitude larger than the 100 MHz choice. The effect of frequency choice on the binding of an analyte on the wall of the well were assessed. To test this, 200 μL goat anti-chicken IgY-HRP solution (immunoglobulin Y with horseradish peroxidase diluted in PBS, ab7118, Abcam, Waltham, MA, USA) was added to the well, then the SAW was applied at 40, 60, or 100 MHz for 15 min, before finally TMB (3,3′,5,5′ tetramethylbenzidine, ab171522, Abcam) was added to incubate for 5 min (with or without SAW). Plotted in FIG. 4, the absorbance intensity with and without SAW indicates the effectiveness of the binding at the three frequencies. The results suggest that 60 MHz is superior to the other two frequency choices, though a check of the data using two-way ANOVA (Analysis of Variance, p=0.141) shows no significant difference between 40 MHz and 60 MHz. In the work that follows, 60 MHz was used since it shows slightly superior performance overall.

FIG. 4 illustrates a bar graph comparing binding efficiency for different SAW frequencies, according to some embodiments of the present disclosure. Specifically, FIG. 4 shows binding efficiency in terms of light absorbance plotted with respect to the application of 40, 60, and 100 MHz SAW at a particle velocity of 96 mm/s (see §). The 96-well plates were coated with 200 μL of the goat anti-chicken IgY-HRP (250 ng/mL) in PBS with SAW for 15 min. Subsequently, TMB was incubated in the well for 5 min without SAW. The numbers at the bottom of each bar are the maximum temperature in the sample, in Celsius, during each run; with SAW, the temperature increased from 25 to 34.2° C. in 15 min. For the no-SAW condition, the ELISA was run at 35° C. from the start. Error bars represent the standard deviation of 6 independent trials. All data was confirmed to be normally distributed via the Shapiro-Wilk test; **p<0.01, ***p<0.001.

The binding assay followed a general ELISA protocol, with each well in a 96-well plate coated with 200 μL of anti-protein A antibody (5 μg/mL; ab19483, Abcam, Waltham, MA, USA) in phosphate-buffered saline (PBS, ThermoFisher Scientific, Waltham, MA, USA) for 1 h at 35° C., washed three times with PBS, then blocked with 200 μL of 1× blocking buffer (ab126587, Abcam) in PBS for 1 h at 35° C. and again washed three times with PBS. Then, a 200 μL goat anti-chicken IgY-HRP solution was added to each well. Some of the wells were mixed using SAW over a range of times from 1 to 20 min. The no-SAW control wells were left at room temperature (25° C.) in the same well tray for the same period of time. The goat anti-chicken IgY-HRP solution was then removed after the mixing and the wells were washed three times with PBS. To achieve a quantitative characterization of the binding through a color change, 100 μL of TMB solution as a suitable reactant with HRP was added into the well. After 10 min incubation, 100 μL stop solution (ab171529, Abcam) was added in the well to stop the reaction. The optical density results were measured at a wavelength of 450 nm with a microplate reader (BioTek Synergy H1, Agilent, Santa Clara, CA, USA). The background absorbance was subtracted from all reported values.

Bead-based ELISA was performed by applying uniform ø2.8 μm superparamagnetic Dynabeads with recombinant Protein A (~45 kDa) covalently coupled to their surface (ThermoFisher Scientific). The well was first pre-treated with 200 μL 1× blocking buffer diluted in PBS for 1 h at 35° C. and then washed three times with PBS. This step was not explicitly needed by the protocol but was used to avoid background noise. Then 2.5 μL of Dynabeads (30 mg/mL) was then added into the well and washed with 50 μL of PBS. The PBS was removed by using a magnet to attract the Dynabeads onto a wall of the well. Then 200 μL anti-protein A antibody (5 μg/mL) was added to incubate with the Dynabeads while being rotary mixed at 300 rpm and 26° C. for 10 min (ThermoMixer C, Eppendorf, Enfield, CT, USA). Placing the magnet near the well once again to trap the magnetic microparticles, the supernatant with unbonded antibodies was poured out and the beads and well were washed thrice with PBS. Subsequently, 200 μL goat anti-chicken IgY-HRP (10 ng/mL) in PBS was then added to each well. One half of the wells were exposed to SAW from 2 to 10 min; the remaining wells were left unexposed to SAW as a control. All the wells were then washed three times in PBS, followed by the addition of 100 μL TMB solution to incubate the system for 10 min without SAW and to create the color change. Finally, 100 μL of stop solution was used to stop the reaction.

To test the effect of the Rayleigh SAW on the antigen-antibody binding, the anti-protein A antibody was immobilized in the wells of the 96-well plate and added 200 μL of goat anti-chicken IgY-HRP at 10 ng/mL. The 60 MHz, 0.8 W, 192 mm/s SAW was applied at this step to mix the solution for a duration of 1 to 15 min, producing both mixing and heating. While the heating does affect the antigen-antibody binding, the temperature increase generated by SAW is less than 35° C. after being applied for 20 min. Notably, the traditional protocol heats the wells to 35° C. for 1 h, and so heating is an inherent part of the protocol.

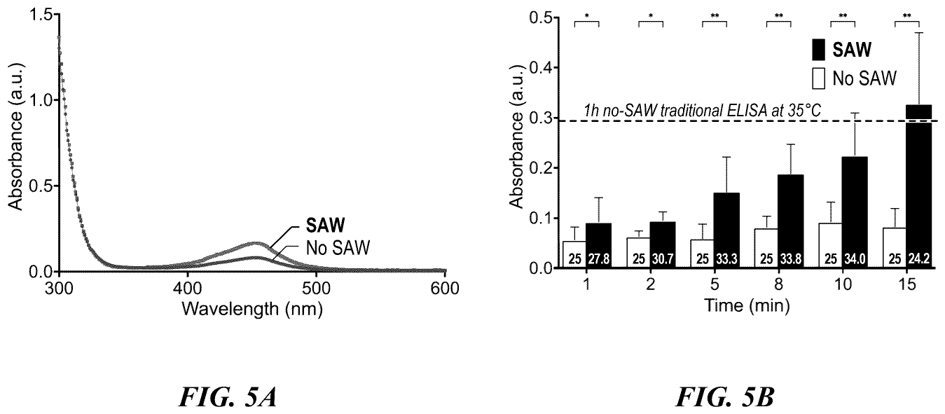

The solution develops a blue color in response to the binding of the secondary antibody to the first, precipitating a reaction of HRP with the added TMB. By adding the stop solution, the enzyme reaction was stopped and turned color from blue to yellow. The strength of the coloration, quantified by the absorbance of light passing through the sample in the wavelength of 450 nm, defines the extent of binding in the sample. FIG. 5A shows an example of the absorbance curves obtained after 1 min of goat anti-chicken IgY-HRP incubation in the well. The maximum intensity was 0.083 and 0.168 for the control (no-SAW) and SAW-driven samples, respectively. FIG. 3B plots the absorbance of the samples after an elapsed time of 1 to 15 min with and without SAW. The horizontal dashed line indicates the absorbance from ELISA after 1 h without SAW at 35° C. in this step. After 1 or 2 min, the binding efficiency indicated by the absorbance value is poor with or without SAW, but nonetheless using SAW significantly (p<0.05) increases the absorbance over the no-SAW controls. After 1 min, SAW improves the absorbance by 51%. This trend continues throughout. As the elapsed time is increased from 5 to 15 min as shown in FIG. 5B, SAW produces a roughly linear improvement in the absorbance, growing to exceed the traditional 1 h no-SAW at 35° C. (dotted line) after just 15 min of SAW. It is likewise 294% greater (p<0.01) than the absorbance of the no-SAW ELISA run for the same amount of time at a lab temperature of 25° C. As a result of faster binding, it took only 10 min to achieve the same binding result as the standard 35° C. ELISA after 1 h. After waiting 10 min with SAW, one can obtain an absorbance of twice the ELISA result without SAW for 10 min.

FIG. 5A illustrates absorbance plotted with respect to wavelength after 1 min with and without SAW already indicates a difference at about 450 nm, with the SAW producing a greater absorbance value. FIG. 5B illustrates results obtained by repeating the (60 MHz, 0.8 W, 192 mm/s) SAW and no-SAW (control) protocols six times for 1 to 15 min describes the effect of time on the ELISA protocol. For these cases, there was no externally applied heating, and the temperature of the samples was at the lab temperature of 25° C., initially for the SAW-driven samples, and throughout for the no-SAW (control) samples. The application of SAW also heats the samples, as indicated by the temperature values provided at the bottom of each bar. However, the heating is generally less than the standard protocol: heating of the sample to 35° C. As a basis for comparison, the dotted line represents the absorbance via the standard ELISA protocol of 1 h at 35° C. without SAW. Error bars represent the standard deviation of 6 independent trials. All data was confirmed to be normally distributed via the Shapiro-Wilk test; *p<0.05, **p<0.01.

While there is a correlation between the absorbance and the maximum measured temperature of each sample, the absorbance value does not correspond to the maximum temperature value measured while applying SAW, indicating that the effects of the SAW are not solely due to temperature increases alone. From 1 to 5 min of SAW, the maximum sample temperature increases by 5° C. and the absorbance improves from 0.093 to 0.15. However, from 5 to 15 min of SAW, the maximum sample temperature only increases by 1° C. while the absorbance nearly doubles from 0.15 to 0.33.

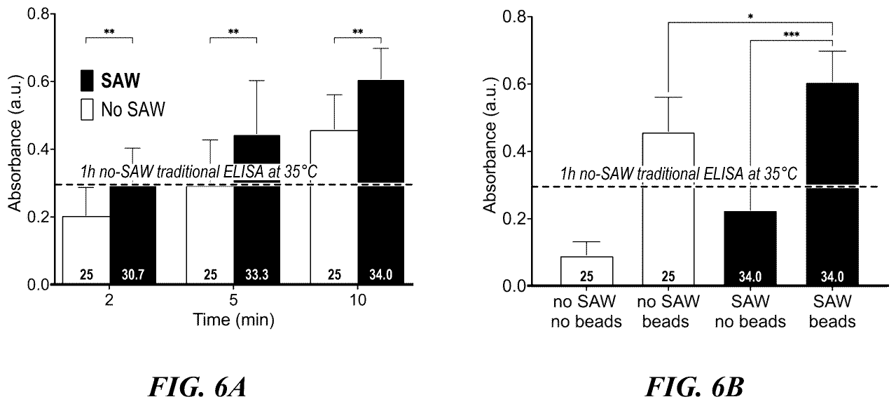

Functionalized beads are also commonly used in ELISA, providing increased surface area and mixing of the beads in the solution, in turn leading to greater sensitivity and, for that matter, multiplexing capabilities. It was also explored whether SAW can enhance binding in the bead-based ELISA system. Using ferromagnetic 2.8 μm diameter Dynabeads at a concentration of 0.375 mg/mL, SAW was applied at the antibody-antigen binding step for 2 to 10 min. As expected, using Dynabeads produces a faster ELISA: Dynabeads reduces the time needed to produce the same absorbance from 1 h (dotted line) to 5 min (FIG. 6A). However, using 60 MHz, 192 mm/s, 0.8 W SAW with the Dynabeads reduces the time necessary to perform the ELISA to just 2 min.

Put another way, in the bead-based approach to ELISA, SAW stimulation is found to significantly (FIG. 6B; p<0.05) increase the absorbance compared to the no-SAW, bead-based control after 10 min incubation. Using Dynabeads also significantly improves the absorbance value (FIG. 6B; p<0.001). By combining the accelerated binding kinetics from SAW and the transport of the roughened binding surface in the form of Dynabeads through SAW-driven mixing, the absorbance was found to be over six times greater than the no-SAW, no-bead control after 10 min incubation. Moreover, after just 10 min, the combination of SAW and Dynabeads produces an absorbance value twice the 1 h no-SAW, no-bead result (FIG. 6A).

FIG. 6A shows that using a bead-based ELISA vastly improves the speed to produce an absorbance comparable to the standard 1-h, no-SAW, 35° C. ELISA protocol (red dashed line) after only 5 min, with 2.5 μL of Dynabeads (30 mg/mL) coated with 200 μL anti-protein A antibody (5 μg/mL) and dilution of the goat anti-chicken IgY-HRP stock solution to 10 ng/ml in PBS for this experiment. Adding 60 MHz, 192 mm/s, 0.8 W SAW to the bead-based ELISA significantly improves the speed to obtain the same results as the traditional protocol after only 2 min. Comparing the results after 10 min incubation, FIG. 6B shows that either introducing beads into the system or introducing SAW into the bead-based system significantly improves the absorbance. As before, the maximum temperature of the samples in Celsius is provided at the bottom of each bar. There was no externally applied heating, and the temperature of the samples was at the lab temperature of 25° C., initially for the SAW-driven samples, and throughout for the no-SAW (control) samples. Error bars represent the standard deviation of 6 independent trials. All data was confirmed to be normally distributed via the Shapiro-Wilk test; *p<0.05, **p<0.01, ***p<0.001.

The above results verified that SAW stimulation can significantly enhance the efficiency of antigen-antibody binding, regardless of whether the antigen/antibody was previously bonded to the well or to the beads. The antigen/antibody may first be bound to the well in traditional ELISA, and this takes considerable time in the overall assay. Even if SAW improves the performance of the antigen-antibody binding, the net benefit of doing so is reduced if the antibody-well wall binding still takes a long time. The effect of SAW on the antibody-well binding as a step in standard ELISA, was examined.

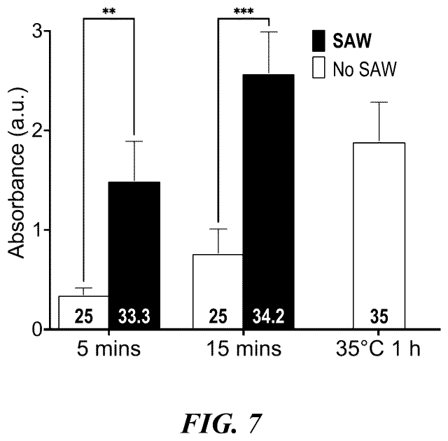

Goat anti-chicken IgY-HRP was used as the surface binding molecule for the well. This allowed utilization of the HRP-TMB reaction as a measure of binding efficiency. As presented in FIG. 7, our findings indicate that SAW stimulation can enhance the antibody-well binding efficiency, producing a two to three-fold increase in efficiency over the no-SAW control. In comparison to the traditional ELISA protocol of waiting 1 h at 35° C. for binding of the antibody to the well, using SAW produces a comparable absorbance after only 15 min.

FIG. 7 illustrates a time course of antibody-well binding with and without 60 MHz, 0.8 W SAW control. 96-well plates were coated with 200 μL of the goat anti-chicken IgY-HRP (250 ng/ml) in PBS at 35° C. for 1 h, room temperature for 5, 15 min and SAW was applied for the indicated time as 5 and 15 min; TMB was then introduced and incubated without SAW for 5 min. Error bars represent a standard deviation of 5 independent trials; *p<0.05, **p<0.01, ***p<0.001. The maximum temperature of the samples in Celsius is provided at the bottom of each bar. There was no externally applied heating, and the temperature of the samples was at the lab temperature of 25° C., initially for the SAW-driven samples, and throughout for the no-SAW (control) samples. Comparing the traditional 35° C., 1-hour ELISA protocol shows that the improvement of the ELISA due to SAW is not due solely to heating.

The ELISA typically involves two to three steps of antigen-antibody binding and antibody-well binding. Through the application of SAW stimulation, our results indicate a reduction in the overall time needed to complete traditional direct sandwich ELISA from approximately 5 h to 1.5 h. Using SAW helps accelerate the binding in both antigen-antibody and antibody-well interactions, thereby streamlining the ELISA process. SAW may also be useful in promoting the speedy deposition of the blocking molecules used to surround the binding sites. However, in our experiments, the use of SAW for this step produced inconclusive results, and so we retained the traditional blocking protocol using blocking buffer in PBS at 35° C. for 1 h, as detailed earlier.

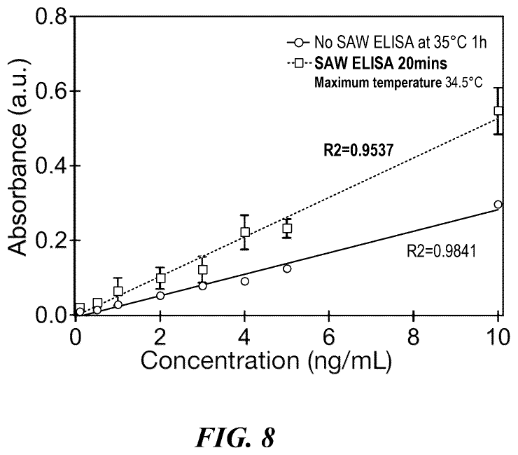

After promising results regarding the time reduction thanks to the SAW, effects on improving the sensitivity were examined. The feasibility of utilizing SAW to facilitate antibody-antigen binding under varying concentrations of the antigen was investigated. The goat anti-chicken IgY-HRP was diluted with PBS to create a concentration range from 0.1 ng/ml to 10 ng/mL, and 0.8 W 60 MHz SAW was applied for a duration of 20 min to ensure sufficient antigen-antibody binding. For both cases (with and without SAW), results indicate that the binding is linearly dependent upon concentration of the antigen with calculated slopes of 0.0289 and 0.0529 for traditional ELISA for 1 h and SAW for 20 min, respectively (FIG. 8). This also demonstrates the improvement in sensitivity of ELISA with SAW stimulation. Notably, under SAW stimulation, the total antigen-antibody binding is found to increase by 83%. Increasing the SAW exposure time to 20 min, causes the total amount of antibody binding to reach equilibrium, the maximum possible in our study. By comparing these results to the traditional 35° C., 1-hour ELISA protocol, it is evident that the improvement in ELISA due to SAW is not simply due to heating, because after 15 min of SAW the sample is at a lower temperature—34.2° C.—yet produces a greater absorbance.

FIG. 8 illustrates the absorbance indicated binding versus target antigen concentration with and without 60 MHz, 0.8 W SAW. SAW improves the absorbance for the same concentration of the target antigen. Error bars represent the standard deviation of 3 independent trials.

The approach disclosed herein offers notable advantages. The total time for the standard ELISA protocol is 3 h at 35° C. or 5 h at room temperature. By using SAW as described here, the complete ELISA protocol can be run at room temperature in 1.5 h. It fits into the normal ELISA workflow, using 96-well trays instead of expecting a laboratory to change its operations to accommodate the technology and to deal with the myriad drawbacks of microfluidics in practical laboratory operations. In the disclosed approach, each well can be individually addressed and controlled by a single IDT in a format compatible with multiplexing to drive all 96 wells at once, in a manner that can be performed using commercially available plates without special training. Past acoustofluidic devices need custom fluidic designs and skilled operators accustomed to acoustofluidics and microfluidics, altogether problematic for adoption in a typical biology lab.

By using the standard bead-based assay, the time needed to perform the ELISA protocol for a useful sensitivity is much shorter, a well-known result. A total of about 20 min is needed for the standard room-temperature assay, including the time to prepare the beads. The beads are ready-to-use with the antibody binding and blocking steps already completed before purchase, and the bead-based kit is expensive as a consequence. Nonetheless, using SAW in the antigen-binding step in combination with the beads halves the time needed for this step to produce a similar absorbance value—from 5 min to 2 min. To be consistent, we retained the 5-min bead resuspension step needed at the beginning of the standard bead-based ELISA protocol. The 1 h blocking step was used for the 96-well tray per the standard protocol to avoid background noise, but this can be done in advance. In any case, it may be that the bead resuspension step may be eliminated, reducing the total time needed for the bead-based ELISA protocol from 20 min to just 2 min.

These improvements may be due to effects beyond SAW-driven heating of the samples. The results show the maximum temperature achieved during SAW use, and in every case the temperature never reached the 35° C. used for the traditional one-hour ELISA protocol, yet still show greater absorbance values after a shorter period of time. For example, after 15 min in FIGS. 5A and 5B. Moreover, there is no direct correlation between the temperature and absorbance across the SAW ELISA tests. The mechanism may rely in part upon another effect.

Notably, diffusion may be responsible for aiding the binding in ELISA in the absence of SAW-driven convection. Antigens near the well walls may quickly bind, leaving a depletion zone that need to be repopulated by additional antigens from the bulk of the fluid sample via diffusion. If the SAW-driven flow was merely laminar, there would be no advantage in driving the flow: the time to bind a given number of antigen molecules from a fluid sample with laminar flow is identical to a fluid sample with no flow at all. However, fast acoustic streaming of the sort present in this system produces non-laminar flow and mixing, suggesting that a combination of convection-dominated binding phenomena together with the mixing of the sample fluid is a significant reason for the improvement in binding seen with the use of SAW.

Acoustic streaming produced by high frequency surface acoustic waves, provides a time-saving and improved sensitivity ELISA that may be directly applied to standard protocols in use today in biology, chemical tooling, and diagnostic laboratories to reduce the time needed for the full protocol from 3-5 h to 1.5 h. It needs essentially no modifications to the existing ELISA protocol. Instead, operators simply place the ELISA plate on top of a SAW module or device disclosed herein and allow acoustic streaming generated in the fluid sample to facilitate faster and more sensitive detection of the target biomarkers. The results show that SAW can be applied in the antibody-well and antigen-antibody binding steps of the protocol, leaving the blocking step unmodified. One may also combine SAW with bead-based ELISA to reduce the total time needed for the antigen-antibody binding step in that protocol from about 10 min to 2 min. The total antigen-antibody binding is increased by 83%, thus increasing the detection sensitivity. With its high sensitivity and efficiency, this approach to ELISA may produce important benefits in the development of diagnostic and therapeutic applications wherever ELISA and its derivatives are used.

The surface acoustic wave (SAW) technology disclosed herein provides technical benefits when applied to ELISA and other fluidic assays, including, for example, acceleration of reaction kinetics, which may significantly reduce assay completion time. In some cases, SAW may reduce assay completion time by at least 10%, 20%, 30%, 40%, 50%, or more, compared to the same assay performed without surface acoustic waves. For example, standard ELISA protocols that typically take 3-5 hours can be completed in approximately 90 minutes using the SAW technology. In some example ELISA applications, binding steps that conventionally take 60 minutes may be reduced to approximately 15 minutes while achieving the same or better binding efficiency. In example bead-based ELISA applications, the binding time can be decreased to as little as 2 minutes, dramatically improving laboratory throughput. See additional description elsewhere in the present document.

In addition to time savings, SAW may enhance sensitivity and overall assay performance. The technology improves detection sensitivity by increasing binding efficiency between antigens and antibodies. In some example ELISA applications, total antigen-antibody binding can be increased by up to 83% compared to traditional methods, resulting in stronger signals and more reliable results. The system provides more consistent and reproducible results through uniform mixing of reagents and samples, achieving higher signal-to-noise ratios in detection measurements.

The effectiveness of the SAW technology may stem from its unique dual mechanism advantage. The technology combines the beneficial effects of both acoustic streaming and controlled heating. Acoustic streaming creates multi-scale fluid flows that substantially enhance molecular transport, overcoming the diffusion limitations inherent in conventional assays. The non-laminar flow patterns, characterized by Reynolds numbers of 1,000-10,000, dramatically improve mixing efficiency by creating complex flow structures that increase the probability of molecular interactions. Simultaneously, the controlled heating supports desirable optimal reaction temperatures without the need of external heating equipment.

Precision control capabilities represent another benefit of the SAW technology. The system features an adjustable duty cycle (e.g., 5%-100%) using pulse width modulation, which allows fine-tuning of the balance between heating and mixing effects for optimal results. The controller (e.g., controller 150) can selectively activate a SAW module during specific assay steps, providing optimization for each stage of the protocol. The operation frequency can be tailored (e.g., in the order of 10 MHz to 100 MHz) for specific applications, and the entire system is customizable for different well configurations, fluid volumes, and assay needs.

The technical effects of SAW technology are achieved in a non-contact manner, meaning that no reagents or particles need to be introduced into the fluid sample for SAW-induced effects to occur. Instead, SAW generates acoustic waves that propagate along a substrate and couple into the fluid, inducing various physical phenomena such as acoustic streaming, microstreaming, and localized heating. These effects enhance fluid mixing, improve mass transport, and facilitate biomolecular interactions without the need for additional chemical modifications or external mechanical components. This non-contact and non-invasive approach preserves the integrity of delicate biological or chemical samples, reduces the risk of contamination, and enables precise control over microfluidic processes. By inducing effects without direct interaction or the need for additional reagents, SAW technology obviates post-processing steps such as sample separation or washing, further streamlining assay workflows. These advantages make SAW highly suitable for applications in biosensing, diagnostics, and lab-on-a-chip platforms, where efficiency, sensitivity, and minimal sample handling are desired.

A practical advantage is the technology's integration and compatibility with existing laboratory infrastructure. The SAW module works seamlessly with standard laboratory equipment and consumables such as 96-well plates, taking essentially no or minimal modification to existing workflows or protocols. This eliminates the need for specialized microfluidic components or custom labware that many alternative technologies would need. The system integrates easily with existing microplate readers and ELISA systems, making adoption straightforward for laboratories.

The benefits of the SAW technology extend beyond ELISA to numerous other fluidic assay applications. The system enhances bead-based assays and prevents nanobead agglomeration, which has been a limiting factor in nanobead assay development. It improves PCR efficiency through better thermal transfer and reagent mixing. Cell culture processes benefit from improved nutrient distribution and waste removal. Crystallization processes are accelerated through improved mass transport, while separation and purification processes gain from gentle but effective mixing that doesn't damage sensitive molecules.

As a further example, the SAW technology disclosed herein may be incorporated into CRISPR-Cas assays to enhance their performance by improving one or multiple steps in the CRISPR-Cas assays. SAW may be applied during key binding steps in CRISPR-Cas assays, which may enhance the binding efficiency between Cas proteins and their target nucleic acids and/or improve the interaction between crRNA and target sequences. In sample preparation, SAW may be used to assist in lysing cells or viral particles, and facilitate a more efficient releasing of nucleic acids for subsequent CRISPR-based detection. The heat generated during an SAW application may enhance the efficiency of isothermal amplification methods, leading to faster and more sensitive detection. SAW may be incorporated into microfluidic CRISPR-Cas detection platforms, such as the electrokinetic microfluidic device mentioned in the review. This may assist with fluid handling, mixing, and potentially concentrate target molecules. For readout systems using fluorescence or colorimetric detection, SAW might be applied to enhance signal generation or improve the distribution of cleaved reporter molecules. In multiplexed CRISPR assays like CARMEN (Combinatorial Arrayed Reactions for Multiplexed Evaluation of Nucleic acids), SAW may potentially assist in droplet manipulation, merging, or mixing within microwell arrays. The heat generated during an SAW application may enhance the efficiency of isothermal amplification methods, leading to faster and more sensitive detection.

As a still further example, the SAW technology disclosed herein may be incorporated into aptamer-based assays to enhance their performance by improving one or multiple steps in the assays. SAW may enhance interaction between aptamers and their targets by improving mass transport and reducing diffusion limitations, thereby reducing assay times. SAW may facilitate efficient mixing of samples and reagents in aptamer-based microfluidic platforms. SAW may enhance non-enzyme mediated signal amplification strategies like hybridization chain reaction (HCR) and catalyzed hairpin assembly (CHA) by accelerating the hybridization process between complementary DNA strands, potentially increasing reaction efficiency. It may assist in sample preparation by facilitating cell lysis for releasing intracellular targets prior to aptamer binding, SAW integration may provide controlled heating beneficial for isothermal amplification reactions. SAW may improve the interaction between aptamer-functionalized nanomaterials and target molecules or assist in their controlled aggregation and dispersion for colorimetric detection methods for signal enhancement.

The technology offers substantial cost and resource benefits for laboratories. Reagent consumption can be reduced through improved binding efficiency, as less material is needed to achieve equivalent or better results. Labor costs associated with lengthy protocols decrease significantly due to shorter assay times. Equipment utilization is extended through faster assay completion, allowing more tests to be run on the same instruments. The system also eliminates the need for chemical additives like surfactants that were previously used to enhance binding, simplifying protocols and reducing potential interference.

These comprehensive technical benefits combine to create a significant advancement in assay technology that addresses the fundamental limitation of diffusion-based binding in traditional assays while maintaining full compatibility with existing laboratory infrastructure and workflows.

The following examples are illustrative of several embodiments of the present technology:

Solution 1. A surface acoustic wave (SAW) facilitated enzyme-linked immunosorbent assay (ELISA) system, comprising:
a piezoelectric substrate;
a transducer assembly including a plurality of transducers that are deposited on the piezoelectric substrate, the transducer assembly configured to apply alternating electric fields to the piezoelectric substrate to induce surface acoustic waves; and
a well plate configured to be positioned to receive the surface acoustic waves from the piezoelectric substrate, the well plate comprising a plurality of wells configured to contain fluid samples relating to an ELISA protocol, wherein the plurality of transducers are arranged to align with the plurality of wells to allow the surface acoustic waves to propagate into the fluid samples in the wells, thereby facilitating performance of the ELISA protocol.

Solution 2. The system of one or more of Solution 1 or any other solutions disclosed herein, wherein each of the plurality of transducers of the transducer assembly is an interdigital transducer (IDT) comprising a plurality of electrode fingers arranged in an interdigitated pattern.

Solution 3. The system of one or more of Solution 2 or any other solutions disclosed herein, wherein the IDT comprises a pair of bus bars with the plurality of electrode fingers extending between the pair of bus bars.

Solution 4. The system of one or more of Solution 3 or any other solutions disclosed herein, wherein the pair of bus bars comprise a first bus bar connected to a signal source and a second bus bar connected to ground.

Solution 5. The system of one or more of Solution 3 or any other solutions disclosed herein, wherein the pair of bus bars have a stepped structure configured to maintain uniform electrical impedance across the plurality of electrode fingers.

Solution 6. The system of one or more of Solution 2 or any other solutions disclosed herein, wherein: each of the plurality of the transducers is positioned at a particular distance from a corresponding well of the plurality of wells to allow the corresponding well to receive surface acoustic waves induced by the corresponding transducer. In some embodiments, each of the plurality of transducers is positioned below a corresponding well.

Solution 7. The system of one or more of Solution 1 or any other solutions disclosed herein, wherein the transducer assembly further comprises at least one reflector positioned to direct the surface acoustic waves in a predetermined direction.

Solution 8. The system of one or more of Solution 7 or any other solutions disclosed herein, wherein the at least one reflector comprises a plurality of electrically disconnected electrode fingers.

Solution 9. The system of one or more of Solution 1 or any other solutions disclosed herein, wherein the piezoelectric substrate is configured to generate Rayleigh surface acoustic waves induced by the applied alternating electric fields.

Solution 10. The system of one or more of Solution 1 or any other solutions disclosed herein, wherein the transducer assembly is configured to operate at a frequency between 5 MHz and 2 GHz.

Solution 11. The system of one or more of Solution 10 or any other solutions disclosed herein, wherein the transducer assembly is configured to operate at approximately 60 MHz.

Solution 12. The system of one or more of Solution 1 or any other solutions disclosed herein, wherein the transducer assembly is configured to operate in a pulsed mode with a variable duty cycle to control a ratio of heating to acoustic streaming in the fluid sample.

Solution 13. The system of one or more of Solution 12 or any other solutions disclosed herein, wherein the transducer assembly is configured to operate using pulse width modulation with an adjustable duty cycle to control a ratio of heating to acoustic streaming in the fluid sample.

Solution 14. The system of one or more of Solution 12 or any other solutions disclosed herein, wherein the pulsed mode comprises alternating between periods when the transducer assembly is energized and periods when the transducer is not energized.

Solution 15. The system of one or more of Solution 14 or any other solutions disclosed herein, further comprising a signal generator configured to provide a pulsed electrical signal to the transducer assembly, wherein a duty cycle of the pulsed electrical signal is adjustable between 5% and 100%.

Solution 16. The system of one or more of Solution 1 or any other solutions disclosed herein, wherein the piezoelectric substrate comprises lithium niobate.

Solution 17. The system of one or more of Solution 16 or any other solutions disclosed herein, wherein the piezoelectric substrate comprises a 127.86° Y-rotated cut lithium niobate.

Solution 18. The system of one or more of Solution 1 or any other solutions disclosed herein, wherein the piezoelectric substrate has a thickness of approximately 500 micrometers.

Solution 19. The system of one or more of Solution 1 or any other solutions disclosed herein, further comprising an acoustic coupling medium disposed between the piezoelectric substrate and the well plate.

Solution 20. The system of one or more of Solution 19 or any other solutions disclosed herein, wherein the acoustic coupling medium facilitates transmission of surface acoustic waves from the piezoelectric substrate to the well plate while minimizing energy loss.

Solution 21. The system of one or more of Solution 1 or any other solutions disclosed herein, wherein the well plate is positioned above the piezoelectric substrate.

Solution 22. The system of one or more of Solution 1 or any other solutions disclosed herein, wherein the well plate comprises a standard 96-well plate format.

Solution 23. The system of one or more of Solution 1 or any other solutions disclosed herein, wherein the well plate comprises a bottom surface that receives the surface acoustic waves from the piezoelectric substrate.

Solution 24. The system of one or more of Solution 23 or any other solutions disclosed herein, wherein: the bottom surface of the well plate is positioned relative to the piezoelectric substrate at a separation distance that enables efficient transfer of acoustic energy into the fluid sample, and the separation distance is occupied by an acoustic coupling medium.

Solution 25. The system of one or more of Solution 24 or any other solutions disclosed herein, wherein the acoustic coupling medium comprises an acoustic gel or a fluid.

Solution 26. The system of one or more of Solution 1 or any other solutions disclosed herein, wherein the well plate is removeably positioned relative to the piezoelectric substrate to allow for replacement of the well plate.

Solution 27. A method of facilitating an enzyme-linked immunosorbent assay (ELISA), comprising:
providing a transducer assembly that comprises at least one transducer deposited on a piezoelectric substrate;
positioning a well plate containing at least one well with a fluid sample on the piezoelectric substrate, the fluid sample relating to an ELISA protocol;
applying an alternating electric field to the transducer assembly to induce surface acoustic waves in the piezoelectric substrate; and
allowing the surface acoustic waves to propagate into the fluid sample to facilitate the ELISA protocol.

Solution 28. The method of one or more of Solution 27 or any other solutions disclosed herein, wherein applying the alternating electric field comprises operating the transducer assembly at a frequency between 5 MHz and 2 GHz.

Solution 29. The method of one or more of Solution 28 or any other solutions disclosed herein, wherein applying the alternating electric field comprises operating the transducer assembly at approximately 60 MHz.

Solution 30. The method of one or more of Solution 27 or any other solutions disclosed herein, wherein applying the alternating electric field comprises operating the transducer assembly in a pulsed mode with a variable duty cycle to control a ratio of heating to acoustic streaming in the fluid sample.

Solution 31. The method of one or more of Solution 30 or any other solutions disclosed herein, wherein operating the transducer assembly in the pulsed mode comprises using pulse width modulation with an adjustable duty cycle.

Solution 32. The method of one or more of Solution 30 or any other solutions disclosed herein, wherein operating the transducer assembly in the pulsed mode comprises setting a duty cycle between 5% and 100%.

Solution 33. The method of one or more of Solution 27 or any other solutions disclosed herein, wherein positioning the well plate comprises providing an acoustic coupling medium between the piezoelectric substrate and the well plate.

Solution 34. The method of one or more of Solution 27 or any other solutions disclosed herein, wherein the ELISA protocol comprises multiple binding steps, and wherein the surface acoustic waves are selectively applied during at least one binding step of the ELISA protocol.

Solution 35. The method of one or more of Solution 34 or any other solutions disclosed herein, wherein the surface acoustic waves are applied during a primary antibody binding step, an analyte binding step, and/or a secondary antibody binding step. In some embodiments, a blocking step is performed in the absence of the surface acoustic waves.

Solution 36. The method of one or more of Solution 27 or any other solutions disclosed herein, wherein propagating the surface acoustic waves into the fluid sample generates both acoustic streaming and heating within the fluid sample.

Solution 37. The method of one or more of Solution 27 or any other solutions disclosed herein, wherein the ELISA protocol is completed in less than 90 minutes using the surface acoustic waves.

Solution 38. The method of one or more of Solution 27 or any other solutions disclosed herein, wherein the ELISA protocol comprises a bead-based ELISA using functionalized microbeads or nanobeads.

Solution 39. The method of one or more of Solution 38 or any other solutions disclosed herein, wherein the surface acoustic waves prevent agglomeration of nanobeads when nanobeads are used in the ELISA protocol.

Solution 40. The method of one or more of Solution 27 or any other solutions disclosed herein, further comprising:
binding a primary antibody to a surface of the well while applying the surface acoustic waves;
adding a blocking buffer to the well;
adding a sample containing target analyte to the well while applying the surface acoustic waves;
adding a detection antibody to the well while applying the surface acoustic waves; and
adding a substrate solution to the well to generate a detectable signal.

Solution 41. The method of one or more of Solution 27 or any other solutions disclosed herein, further comprising measuring the detectable signal to determine a presence or concentration of the target analyte.

Solution 42. The method of one or more of Solution 27 or any other solutions disclosed herein, wherein the method reduces a time required for the ELISA protocol by at least 50% compared to a standard ELISA protocol performed without surface acoustic waves.

Solution 43. The method of one or more of Solution 27 or any other solutions disclosed herein, further comprising directing the surface acoustic waves in a predetermined direction using at least one reflector positioned adjacent to the at least one transducer.

Solution 44. The method of one or more of Solution 27 or any other solutions disclosed herein, wherein applying the alternating electric field induces Rayleigh surface acoustic waves.

Solution 45. An enzyme-linked immunosorbent assay (ELISA) system, comprising:
a microplate reader; and
a surface acoustic wave (SAW) module configured to be positioned beneath a standard multi-well plate, the SAW module comprising:
   a piezoelectric substrate; and
   a transducer assembly comprising at least one transducer deposited on the piezoelectric substrate and configured to apply alternating electric fields to induce surface acoustic waves, wherein the system is configured to propagate the surface acoustic waves into fluid samples in a standard multi-well plate when the standard multi-well plate is positioned on the SAW module during operation to facilitate or enhance reaction kinetics, including binding kinetics, of an ELISA protocol.

Solution 46. The ELISA system of one or more of Solution 45 or any other solutions disclosed herein, wherein the surface acoustic wave module is removable and configured to be inserted into an existing ELISA system without modification to the microplate reader.

Solution 47. The ELISA system of one or more of Solution 45 or any other solutions disclosed herein, further comprising an acoustic coupling medium applicator configured to apply an acoustic coupling medium between the SAW module and the standard multi-well plate.

Solution 48. The ELISA system of one or more of Solution 45 or any other solutions disclosed herein, further comprising a control unit configured to: synchronize operation of the SAW module with steps of the ELISA protocol; and selectively activate the SAW module during one or more binding steps of the ELISA protocol.

Solution 49. The ELISA system of one or more of Solution 45 or any other solutions disclosed herein, wherein the SAW module is dimensioned to match a footprint of the standard multi-well plate.

Solution 50. The ELISA system of one or more of Solution 45 or any other solutions disclosed herein, wherein the SAW module is integrated into a tray or platform that replaces a standard tray of the microplate reader.

Solution 51. An adapter for facilitating a fluidic assay, comprising:
a piezoelectric substrate configured to be acoustically coupled to a reaction vessel;
a transducer assembly comprising at least one transducer that is deposited on the piezoelectric substrate; and
a signal interface configured to connect to a power source, wherein the adapter is configured to generate surface acoustic waves that propagate into a fluid sample in the reaction vessel to enhance reaction kinetics of the fluidic assay. In some embodiments, the adapter is configured to be incorporated into an existing assay equipment without or with minimal modification of the existing assay equipment.

Solution 52. The adapter of one or more of Solution 51 or any other solutions disclosed herein, further comprising a controller configured to adjust operating parameters of the transducer assembly based on a type of assay being performed.

Solution 53. The adapter of one or more of Solution 52 or any other solutions disclosed herein, wherein the controller is configured to operate the transducer assembly in a pulsed mode with an adjustable duty cycle to control a ratio of heating to acoustic streaming in the fluid sample.

Solution 54. The adapter of one or more of Solution 51 or any other solutions disclosed herein, wherein the transducer assembly comprises an array of transducers arranged to align with different regions of the reaction vessel.

Solution 55. The adapter of one or more of Solution 51 or any other solutions disclosed herein, further comprising one or more alignment features configured to facilitate positioning of the reaction vessel relative to the piezoelectric substrate.

Solution 56. The adapter of one or more of Solution 51 or any other solutions disclosed herein, further comprising a temperature sensor configured to monitor temperature within the reaction vessel during operation.

Solution 57. The adapter of one or more of Solution 51 or any other solutions disclosed herein, wherein the reaction vessel comprises a multi-well plate, a microfluidic chip, a petri dish, a flask, a test tube, or a cuvette.

Solution 58. The adapter of one or more of Solution 51 or any other solutions disclosed herein, wherein the adapter is configured to enhance an enzyme-linked immunosorbent assay (ELISA) performed in the reaction vessel.

Solution 59. The adapter of one or more of Solution 51 or any other solutions disclosed herein, wherein the adapter is configured to enhance polymerase chain reaction (PCR) performed in the reaction vessel.

Solution 60. The adapter of one or more of Solution 51 or any other solutions disclosed herein, wherein the adapter is configured to enhance cell culture processes by improving nutrient mixing and waste product removal.

Solution 61. The adapter of one or more of Solution 51 or any other solutions disclosed herein, wherein the adapter is configured to enhance crystallization processes by improving mass transport and preventing sedimentation.

Solution 62. The adapter of one or more of Solution 51 or any other solutions disclosed herein, wherein the adapter is configured to enhance separation or purification processes within the reaction vessel.

Solution 63. The adapter of one or more of Solution 51 or any other solutions disclosed herein, wherein the adapter is configured to enhance bead-based assays by preventing agglomeration of beads within the fluid sample.

Solution 64. The adapter of one or more of Solution 51 or any other solutions disclosed herein, further comprising a dispenser for automatically applying an acoustic coupling medium between the piezoelectric substrate and the reaction vessel.

Solution 65. The adapter of one or more of Solution 51 or any other solutions disclosed herein, wherein the adapter is configured to reduce assay completion time by at least 50% compared to the same assay performed without surface acoustic waves.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Various embodiments described herein are described in the general context of methods or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), Blu-ray Discs, etc. Therefore, the computer-readable media described in the present application include non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

For example, one aspect of the disclosed embodiments relates to a computer program product that is embodied on a non-transitory computer readable medium. The computer program product includes program code for carrying out any one or and/or all of the operations of the disclosed embodiments.

Only a few implementations and examples are described and other implementations, enhancements and variations can be made based on what is described and illustrated in this patent document.

What is claimed is:

1. A surface acoustic wave (SAW) facilitated enzyme-linked immunosorbent assay (ELISA) system, comprising:
a piezoelectric substrate;
a transducer assembly including a plurality of transducers that are deposited on the piezoelectric substrate, the transducer assembly configured to apply alternating electric fields to the piezoelectric substrate to induce surface acoustic waves; and
a well plate configured to be positioned to receive the surface acoustic waves from the piezoelectric substrate, the well plate comprising a plurality of wells configured to contain fluid samples relating to an ELISA protocol, wherein the plurality of transducers are arranged to align with the plurality of wells to allow the surface acoustic waves to propagate into the fluid samples in the wells, thereby facilitating performance of the ELISA protocol.

2. The system of claim 1, wherein each of the plurality of the transducers is positioned at a particular distance from a corresponding well of the plurality of wells to allow the corresponding well to receive surface acoustic waves induced by the corresponding transducer.

3. The system of claim 1, wherein each of the plurality of transducers is positioned below a corresponding well.

4. The system of claim 1, wherein:
the well plate comprises a bottom surface that receives the surface acoustic waves from the piezoelectric substrate;
the bottom surface of the well plate is positioned relative to the piezoelectric substrate at a separation distance that enables efficient transfer of acoustic energy into the fluid sample; and
the separation distance is occupied by an acoustic coupling medium.

5. The system of claim 1, wherein:
the transducer assembly further comprises at least one reflector that comprises a plurality of electrically disconnected electrode fingers; and
the at least one reflector is positioned to direct the surface acoustic waves in a predetermined direction.

6. The system of claim 1, wherein each of at least one of the plurality of transducers is an interdigital transducer (IDT) comprising a plurality of electrode fingers arranged in an interdigitated pattern.

7. The system of claim 1, wherein the piezoelectric substrate is configured to generate Rayleigh surface acoustic waves induced by the applied alternating electric fields.

8. The system of claim 1, wherein the transducer assembly is configured to operate in a pulsed mode with a variable duty cycle to control a ratio of heating to acoustic streaming in the fluid samples.

9. The system of claim 8, wherein the pulse mode includes pulse width modulation with an adjustable duty cycle.

10. The system of claim 1, wherein the piezoelectric substrate comprises lithium niobate.

11. The system of claim 1, wherein the piezoelectric substrate has a thickness of approximately 500 micrometers.

12. The system of claim 1, wherein the well plate is removeably positioned relative to the piezoelectric substrate to allow for replacement of the well plate.

13. The system of claim 1, wherein the transducer assembly is configured to operate at a frequency between 5 MHz and 2 GHz.

14. A method of facilitating an enzyme-linked immunosorbent assay (ELISA), comprising:
providing a transducer assembly that comprises at least one transducer deposited on a piezoelectric substrate;
positioning a well plate containing at least one well with a fluid sample on the piezoelectric substrate, the fluid sample relating to an ELISA protocol;
applying an alternating electric field to the transducer assembly to induce surface acoustic waves in the piezoelectric substrate; and
allowing the surface acoustic waves to propagate into the fluid sample to facilitate performance of the ELISA protocol.

15. The method of claim 14, wherein applying the alternating electric field comprises operating the transducer assembly in a pulsed mode with a variable duty cycle to control a ratio of heating to acoustic streaming in the fluid sample.

16. The method of claim 14, wherein positioning the well plate comprises providing an acoustic coupling medium between the piezoelectric substrate and the well plate.

17. The method of claim 14, wherein the ELISA protocol comprises multiple binding steps, and wherein the surface acoustic waves are selectively applied during at least one binding step of the ELISA protocol.

18. The method of claim 14, wherein the ELISA protocol comprises a bead-based ELISA using functionalized microbeads or nanobeads.

19. The method of claim 14, further comprising measuring a detectable signal to determine a presence or concentration of a target analyte in the fluid sample.

20. An adapter for facilitating a fluidic assay, comprising:
a piezoelectric substrate configured to be acoustically coupled to a reaction vessel;
a transducer assembly comprising at least one transducer that is deposited on the piezoelectric substrate; and
a signal interface configured to connect the transducer assembly to a power source, wherein the adapter is configured to generate surface acoustic waves that propagate into a fluid sample in the reaction vessel to enhance reaction kinetics of the fluidic assay.
